SEQUENCE LISTING (8 sequences)

SEQ ID NO: 1 - DNA, 21 bp, Artificial Sequence
  primer
ccaccatttctcctgcttgaa

SEQ ID NO: 2 - DNA, 23 bp, Artificial Sequence
  primer
gtcgagtggtctcagttggagat

SEQ ID NO: 3 - DNA, 29 bp, Artificial Sequence
  probe
ctcgttcaatccataaacacgtgcaatcc

SEQ ID NO: 4 - DNA, 75 bp, Artificial Sequence
  amplicon
gtcgagtggtctcagttggagatgggattgcacgtgtttatggattgaacgagattcaagcaggagaaatggtgg

SEQ ID NO: 5 - DNA, 20 bp, Artificial Sequence
  primer
caaggacgccgaattcaaga

SEQ ID NO: 6 - DNA, 20 bp, Artificial Sequence
  primer
cgaagaaggtgcccttgaga

SEQ ID NO: 7 - DNA, 26 bp, Artificial Sequence
  probe
ccacccgatgaacttcctgaacgaga

SEQ ID NO: 8 - DNA, 71 bp, Artificial Sequence
  amplicon
caaggacgccgaattcaagacccacccgatgaacttcctgaacgagaggactctcaagggcaccttcttcg

(19) United States
(12) Patent Application Publication
(10) Pub. No.: US 2020/0236885 A1
(43) Pub. Date: Jul. 30, 2020

(54) HIGH-THROUGHPUT METHOD FOR MITOCHONDRIA ISOLATION FROM PLANT SEEDS

(71) Applicants: Syngenta Participations AG, Basel (CH); SYGENTA CROP PROTECTION AG, Basel (CH)

(72) Inventors: Wenjin YU, Research Triangle Park, NC (US); Chunyang FAN, Research Triangle Park, NC (US); Yanshan JI, Research Triangle Park, NC (US)

(73) Assignee: Syngenta Participations AG, Basel (CH)

(21) Appl. No.: 16/652,574

(22) PCT Filed: Oct. 15, 2018
(86) PCT No.: PCT/US18/55820

§ 371 Date: Mar. 31, 2020

Related U.S. Application Data
(60) Provisional application No. 62/572,780, filed on Oct. 16, 2017.

(51) Int. Cl. A01H 5/10; C12N 15/10; C12Q 1/6806; C12Q 1/6895

(52) U.S. Cl. CPC A01H 5/10; C12Q 1/6895; C12Q 1/6806; C12N 15/1003

(57) ABSTRACT

The invention relates to methods of extracting mitochondrial DNA from whole seeds in a high-throughput environment. The method comprises grinding a population of whole seeds, preferably wheat or barley seeds; isolating the mitochondria from the seeds; and extracting the mitochondrial DNA. Methods also relate to the use of low-speed centrifugation, which permits the methods use in a high-throughput environment.

### FIELD OF THE INVENTION

The presently disclosed subject matter relates to methods of isolating subcellular organelles, particularly mitochondria, from whole seeds. The isolated organelles can be further processed to isolate organellular DNA, which can be subjected to downstream analyses, including real-time PCR, quantitative PCR, SNP discovery and detection, and genotyping.

### BACKGROUND

Heterosis (also known as hybrid vigor) in plants is the primary goal of modern plant breeding. Breeders cross a variety of individual plants in the hope of obtaining progeny hybrid plants which display improved characteristics compared to either parent, i.e., heterosis. These heterosis characteristics include increased yield, increased reproductive ability, increase in size, earlier flowering and maturity, greater resistance to disease and pests, faster growth rate, and others. However, plants are capable of self-fertilization, which leads to progeny inbred plants that are genetically identical to the parent plants. This phenomenon reduces the total number of progeny hybrid plants which can be successfully screened for heterosis.

In order to reduce the frequency of self-fertilization (and increase the number of cross-fertilization events), breeders create physical barriers to self-fertilization. For example, a breeder may plant the parent plants in different plots and hand-carry the pollen from one plant to another. In another alternative, the male reproductive organs are physically removed or chemically rendered inert so as to prevent self-fertilization events. One alternative is to use a cytoplasmic male sterile (“CMS”) system. See, for example, U.S. Pat. No. 3,842,538 (issued Oct. 22, 1974), incorporated herein by reference in its entirety.

Briefly, using a CMS breeding system prevents self-fertilization events from occurring as frequently. A CMS breeding system requires three lines: a mother line, a father line, and a maintainer line. The mother line is cytoplasmically male sterile, conferred by mutations in the mitochondria of the line. In a typical CMS-enabled breeding program, the mother line requires generational maintenance by crossing with a maintainer line, which is not cytoplasmically male sterile but is homozygous recessive for restorer genes. This cross creates next-generation plants comprising the mother line's mitochondria, conferring cytoplasmic male sterility. When the breeder is ready to create a hybrid line, a father line is crossed with the mother line. The father line is at least heterozygous dominant for restorer genes. Because the mother line cannot be self-fertilized, F1 seeds produced must be by a cross with the father line. And because the father line possesses restorer genes, the F1 progeny is both male and female fertile.

There are many reasons beyond CMS breeding systems analyzing the mitochondrial DNA in the absence of genomic DNA, including mitochondrial genome sequencing. In some plant species, particularly wheat, understanding mitochondrial differences at the genetic level is very difficult—or impossible—to determine without first isolating the mitochondria. This is because during the course of evolution mitochondria and host genomes have shared their genetic codes through recombination events and gene transfer. Therefore, to analyze mitochondria requires performing sophisticated tissue separation, mitochondria isolation, and only then performing genetic analysis.

Until this invention, the prior state of the art taught, at a minimum, isolating the embryo from a seed because the remainder of the seed (e.g., the endosperm, pericarp or seed coat, or other seed portions) would interfere with mitochondrial DNA isolation. Additionally, the prior art taught isolating mitochondria organelles using several time-consuming several high-speed centrifugation steps. See, e.g., Zaheer Ahmed and Yong-Bi Fu, An improved method with a wider applicability to isolate plant mitochondria for mtDNA extraction, PLANT METHODS (2015) 11:56. Both prior art requirements prevent the prior art methods from being performed in a high-throughput manner.

### SUMMARY

The present invention significantly improves the art by providing a method for isolating mitochondrial DNA from dry seeds. In one embodiment, the method requires taking a bulk of dry seeds and grinding them into a powder; sampling the powder and contacting the sample of powder with homogenization buffer, and optionally incubating the sample in the buffer; centrifuging the sample, particularly at low speed such as 2000-4000×g, to obtain a supernatant containing plant mitochondria; and treating the supernatant with DNase in order to remove any contaminant genomic DNA. In one aspect, the homogenization buffer comprises Tris and sucrose, and in particular comprises 50 mM Tris-HCl ph 7.5 and 0.5 M sucrose. In another embodiment, mitochondrial DNA is isolated from the plant mitochondria. In particular, the dry seeds used as a starting point are wheat seeds, but they may also be barley seeds, corn seeds, ride seeds, sunflower seeds, or seeds of another crop plant.

In another embodiment, the invention isolates plant mitochondria in a high-throughput manner. This high-throughput method requires taking several dry seed bulks, and grinding them individually into separate powders. From these separate powders, samples are taken, each representing one of the seed bulks, and placed into individual wells of a sampling plate. Homogenization buffer is added to each well of the sampling plate, and the plate is then optionally incubated. The plate (or plates, if more than one) is centrifuged, particularly at a low speed such as 2000-4000×g, to pellet the seed debris and nuclei and thus obtain a supernatant containing plant mitochondria. The supernatant from each well is transferred to a corresponding well in a new sampling plate. Each well in the new plate is treated with DNase in order to remove any contaminant genomic DNA. In one aspect, the homogenization buffer comprises Tris and sucrose, and in particular comprises 50 mM Tris-HCl pH 7.5 and 0.5 M sucrose. In another embodiment, mitochondrial DNA is isolated from the plant mitochondria. In particular, the dry seeds used as a starting point are wheat seeds, but they may also be barley seeds, corn seeds, rice seeds, sunflower seeds, or seeds of another crop plant. In another aspect, the sampling plate is a 24-well plant, or a 48-well plate, or a 96-well plate.

In another embodiment, the invention relates to a method of conducting dual genotyping on the mitochondrial DNA and the genomic DNA obtained from the same sample. Dry seeds are ground as stated above, and plant mitochondrial DNA are obtained as stated above. After transferring the supernatant (containing the plant mitochondria) to a new sampling plate, the precipitated cell debris is resuspended in homogenization buffer. The resuspended cell debris is then processed according to prior art methods in order to extract genomic DNA, which may be achieved through known gDNA extraction methods. See, e.g., Stephen L. Dellaporta, Jonathan Wood, James B. Hicks, A plant DNA minipreparation: Version II, PLANT MOLECULAR BIOLOGY REPORTER, 1983, Volume 1, Issue 4, pp 19-21.

### Definitions

This invention is not limited to the particular methodology, protocols, cell lines, plant species or genera, constructs, and reagents described herein. The terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a plant” is a reference to one or more plants and includes equivalents thereof known to those skilled in the art, and so forth. As used herein, the word “or” means any one member of a particular list and also includes any combination of members of that list (i.e., includes also “and”).

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means ±1° C., preferably ±0.5° C. Where the term “about” is used in the context of this invention (e.g., in combinations with temperature or molecular weight values) the exact value (i.e., without “about”) is preferred.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

The term “genotype” refers to the genetic constitution of a cell or organism. An individual's “genotype for a set of genetic markers” includes the specific alleles, for one or more genetic marker loci, present in the individual. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest (e.g., a quantitative trait as defined herein). Thus, in some embodiments a genotype comprises a sum of one or more alleles present within an individual at one or more genetic loci of a quantitative trait.

The term “isolated,” when used in the context of the nucleic acid molecules or polynucleotides of the present invention, refers to a polynucleotide that is identified within and isolated/separated from its chromosomal polynucleotide context within the respective source organism. An isolated nucleic acid or polynucleotide is not a nucleic acid as it occurs in its natural context, if it indeed has a naturally occurring counterpart. In contrast, non-isolated nucleic acids are nucleic acids such as DNA and RNA, which are found in the state they exist in nature. For example, a given polynucleotide (e.g., a gene) is found on the host cell chromosome in proximity to neighboring genes. The isolated nucleic acid molecule may be present in single-stranded or double-stranded form. Alternatively, it may contain both the sense and antisense strands (i.e., the nucleic acid molecule may be double-stranded). In a preferred embodiment, the nucleic acid molecules of the present invention are understood to be isolated.

The phrase “nucleic acid” or “polynucleotide” refers to any physical string of monomer units that can be corresponded to a string of nucleotides, including a polymer of nucleotides (e.g., a typical DNA polymer or polydeoxyribonucleotide or RNA polymer or polyribonucleotide), modified oligonucleotides (e.g., oligonucleotides comprising bases that are not typical to biological RNA or DNA, such as 2′-O-methylated oligonucleotides), and the like. In some embodiments, a nucleic acid or polynucleotide can be single-stranded, double-stranded, multi-stranded, or combinations thereof. Unless otherwise indicated, a particular nucleic acid or polynucleotide of the present invention optionally comprises or encodes complementary polynucleotides, in addition to any polynucleotide explicitly indicated.

“PCR (polymerase chain reaction)” is understood within the scope of the invention to refer to a method of producing relatively large amounts of specific regions of DNA, thereby making possible various analyses that are based on those regions.

The term “probe” refers to a single-stranded oligonucleotide that will form a hydrogen-bonded duplex with a substantially complementary oligonucleotide in a target nucleic acid analyte or its cDNA derivative.

The term “primer”, as used herein, refers to an oligonucleotide which is capable of annealing to the amplification target allowing a DNA polymerase to attach, thereby serving as a point of initiation of DNA synthesis when placed under conditions in which synthesis of primer extension product is induced, e.g., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer is generally sufficiently long to prime the synthesis of extension products in the presence of the agent for polymerization. The exact lengths of the primers will depend on many factors, including temperature and composition (A/T and G/C content) of primer. A pair of bi-directional primers consists of one forward and one reverse primer as commonly used in the art of DNA amplification such as in PCR amplification. It will be understood that “primer,” as used herein, may refer to more than one primer, particularly in the case where there is some ambiguity in the information regarding the terminal sequence(s) of the target region to be amplified. Hence, a “primer” includes a collection of primer oligonucleotides containing sequences representing the possible variations in the sequence or includes nucleotides which allow a typical base pairing. The oligonucleotide primers may be prepared by any suitable method. Methods for preparing oligonucleotides of specific sequence are known in the art, and include, for example, cloning and restriction of appropriate sequences, and direct chemical synthesis. Chemical synthesis methods may include, for example, the phospho di- or tri-ester method, the diethylphosphoramidate method and the solid support method disclosed in, for example, U.S. Pat. No. 4,458,066. The primers may be labeled, if desired, by incorporating means detectable by, for instance, spectroscopic, fluorescence, photochemical, biochemical, immunochemical, or chemical means. Template-dependent extension of the oligonucleotide primer(s) is catalyzed by a polymerizing agent in the presence of adequate amounts of the four deoxyribonucleotide triphosphates (dATP, dGTP, dCTP and dTTP, i.e. dNTPs) or analogues, in a reaction medium which is comprised of the appropriate salts, metal cations, and pH buffering system. Suitable polymerizing agents are enzymes known to catalyze primer- and template-dependent DNA synthesis. Known DNA polymerases include, for example, E. coli DNA polymerase I or its Klenow fragment, T4 DNA polymerase, and Taq DNA polymerase. The reaction conditions for catalyzing DNA synthesis with these DNA polymerases are known in the art. The products of the synthesis are duplex molecules consisting of the template strands and the primer extension strands, which include the target sequence. These products, in turn, serve as template for another round of replication. In the second round of replication, the primer extension strand of the first cycle is annealed with its complementary primer; synthesis yields a “short” product which is bound on both the 5′- and the 3′-ends by primer sequences or their complements. Repeated cycles of denaturation, primer annealing, and extension result in the exponential accumulation of the target region defined by the primers. Sufficient cycles are run to achieve the desired amount of polynucleotide containing the target region of nucleic acid. The desired amount may vary, and is determined by the function which the product polynucleotide is to serve. The PCR method is well described in handbooks and known to the skilled person. After amplification by PCR, the target polynucleotides may be detected by hybridization with a probe polynucleotide which forms a stable hybrid with that of the target sequence under low, moderate, or even highly stringent hybridization and wash conditions. If it is expected that the probes will be essentially completely complementary (i.e., about 99% or greater) to the target sequence, highly stringent conditions may be used. If some mismatching is expected, for example if variant strains are expected with the result that the probe will not be completely complementary, the stringency of hybridization may be lessened. However, conditions are typically chosen which rule out nonspecific/adventitious binding. Conditions, which affect hybridization, and which select against nonspecific binding are known in the art, and are described in, for example, Sambrook and Russell, 2001. Generally, lower salt concentration and higher temperature increase the stringency of hybridization conditions. “PCR primer” is preferably understood within the scope of the present invention to refer to relatively short fragments of single-stranded DNA used in the PCR amplification of specific regions of DNA.

The terms “protein,” “peptide” and “polypeptide” are used interchangeably herein.

The term “allele(s)” means any of one or more alternative forms of a gene, all of which alleles relate to at least one trait or characteristic. In a diploid cell, the two alleles of a given gene occupy corresponding loci on a pair of homologous chromosomes. In some instances (e.g., for QTLs) it is more accurate to refer to “haplotype” (i.e., an allele of a chromosomal segment) instead of “allele”, however, in those instances, the term “allele” should be understood to comprise the term “haplotype”. If two individuals possess the same allele at a particular locus, the alleles are termed “identical by descent” if the alleles were inherited from one common ancestor (i.e., the alleles are copies of the same parental allele). The alternative is that the alleles are “identical by state” (i.e., the alleles appear to be the same but are derived from two different copies of the allele). Identity by descent information is useful for linkage studies; both identity by descent and identity by state information can be used in association studies, although identity by descent information can be particularly useful.

The term “backcrossing” is understood within the scope of the invention to refer to a process in which a hybrid progeny is repeatedly crossed back to one of the parents.

The term “conditionally male sterile” means a phenotype of male sterility (i.e., an incapability to produce fertile pollen), which can be induced and/or repressed by certain conditions. In consequence, a plant can be “switched” from a male sterile to a male fertile phenotype by applying said certain conditions. Male sterility can be caused by various factors and can be expressed for example as a complete lack of male organs (anthers), degenerated pollen, infertile pollen etc. Based on the intensity of the condition the “switch” from male sterility to male fertility may be complete or incomplete. Most preferably, in the context of the present invention the term “conditionally male sterile” means a temperature-dependent male sterility and thereby means a nuclear male sterile phenotype, wherein the sterility is temperature de-pendent and can be reverted to fertility at a temperature of more than 35° C. (preferably between 35° C. and 43° C., more preferably between 37° C. and 40° C., most preferably at about 39° C.; preferably with an exposure for a preferred heat treatment time and a subsequent growing at ambient temperature).

The term “germplasm” refers to the totality of the genotypes of a population or another group of individuals (e.g., a species). The term “germplasm” can also refer to plant material; e.g., a group of plants that act as a repository for various alleles. The phrase “adapted germplasm” refers to plant materials of proven genetic superiority; e.g., for a given environment or geo-graphical area, while the phrases “non-adapted germplasm”, “raw germplasm”, and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

The term “haplotype” can refer to the set of alleles an individual inherited from one parent. A diploid individual thus has two haplotypes. The term “haplotype” can be used in a more limited sense to refer to physically linked and/or unlinked genetic markers (e.g., sequence polymorphisms) associated with a phenotypic trait. The phrase “haplotype block” (sometimes also referred to in the literature simply as a haplotype) refers to a group of two or more genetic markers that are physically linked on a single chromosome (or a portion thereof). Typically, each block has a few common haplotypes, and a subset of the genetic markers (i.e., a “haplo-type tag”) can be chosen that uniquely identifies each of these haplotypes.

The terms “hybrid”, “hybrid plant”, and “hybrid progeny” in the context of plant breeding refer to a plant that is the offspring of genetically dissimilar parents produced by crossing plants of different lines or breeds or species, including but not limited to the cross between two inbred lines (e.g., a genetically heterozygous or mostly heterozygous individual). The phrase “single cross F1 hybrid” refers to an F1 hybrid produced from a cross between two inbred lines.

The phrase “inbred line” refers to a genetically homozygous or nearly homozygous population. An inbred line, for example, can be derived through several cycles of brother/sister breedings or of selfing. In some embodiments, inbred lines breed true for one or more phenotypic traits of interest. An “inbred”, “inbred individual,” or “inbred progeny” is an individual sampled from an inbred line. The term “inbred” means a substantially homozygous individual or line.

The terms “introgression,” “introgressed,” and “introgressing” refer to both a natural and artificial process whereby genomic regions of one species, variety, or cultivar are moved into the genome of another species, variety, or cultivar, by crossing those species. The process may optionally be completed by backcrossing to the recurrent parent.

The term “marker-based selection” is understood within the scope of the invention to refer to the use of genetic markers to detect one or more nucleic acids from the plant, where the nucleic acid is associated with a desired trait to identify plants that carry genes for desirable (or undesirable) traits, so that those plants can be used (or avoided) in a selective breeding program.

The phrase “phenotypic trait” refers to the appearance or other detectable characteristic of an individual, resulting from the interaction of its genome with the environment.

The term “plurality” refers to more than one entity. Thus, a “plurality of individuals” refers to at least two individuals. In some embodiments, the term plurality refers to more than half of the whole. For example, in some embodiments a “plurality of a population” refers to more than half the members of that population.

The term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

The phrase “qualitative trait” refers to a phenotypic trait that is controlled by one or a few genes that exhibit major phenotypic effects. Because of this, qualitative traits are typically simply inherited. Examples in plants include, but are not limited to, flower color, cob color, and disease resistance such as for example Northern corn leaf blight resistance.

“Phenotype” is understood within the scope of the invention to refer to a distinguishable characteristic(s) of a genetically controlled trait.

A “plant” is any plant at any stage of development, particularly a seed plant.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any group of plant cells organized into structural and/or functional units. The use of this term in conjunction with, or in the absence of, any specific type of plant tissue as listed above or otherwise embraced by this definition is not intended to be exclusive of any other type of plant tissue.

The term “plant part” indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as pollen, ovules, leaves, embryos, roots, root tips, anthers, flowers, fruits, stems, shoots, scions, rootstocks, seeds, protoplasts, calli, and the like.

The term “population” means a genetically heterogeneous collection of plants sharing a common genetic derivation.

The term “predominately male sterile” means that in a population of at least 100 plants not more than 10%, preferably not more than 5%, more preferably not more than 1% of the flowers on all of those plants have functional male organs producing fertile pollen. It has to be understood that an individual plant can have both fertile and sterile flowers. In preferred embodiments not more than 10%, preferably not more than 5%, more preferably not more than 1% of the flowers on an individual plant have functional male organs producing fertile pollen.

The term “offspring” plant refers to any plant resulting as progeny from a vegetative or sexual reproduction from one or more parent plants or descendants thereof. For instance, an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait, while offsprings of second generation (F2) or subsequent generations (F3, F4, etc.) are specimens produced from selfings of F1's, F2's etc. An F1 may thus be a hybrid resulting from a cross between two true breeding parents (true-breeding is homo-zygous for a trait), while an F2 may be an offspring resulting from self-pollination of said F1 hybrids.

“Recombination” is the exchange of information between two homologous chromosomes during meiosis. The frequency of double recombination is the product of the frequencies of the single recombinants. For instance, a recombinant in a 10 cM area can be found with a frequency of 10%, and double recombinants are found with a frequency of 10%×10%=1% (1 centimorgan is defined as 1% recombinant progeny in a testcross).

The term “RHS” or “restored hybrid system” means a nuclear male sterility based hybrid system.

The phrases “sexually crossed” and “sexual reproduction” in the context of the present invention refer to the fusion of gametes to produce progeny (e.g., by fertilization, such as to produce seed by pollination in plants). In some embodiments, a “sexual cross” or “cross-fertilization” is fertilization of one individual by another (e.g., cross-pollination in plants). In some embodiments the term “selfing” refers to the production of seed by self-fertilization or self-pollination; i.e., pollen and ovule are from the same plant.

“Selective breeding” is understood within the scope of the present invention to refer to a program of breeding that uses plants that possess or display desirable traits as parents.

“Tester plant” is understood within the scope of the present invention to refer to a plant used to characterize genetically a trait in a plant to be tested. Typically, the plant to be tested is crossed with a “tester” plant and the segregation ratio of the trait in the progeny of the cross is scored.

The term “tester” refers to a line or individual with a standard genotype, known characteristics, and established performance. A “tester parent” is an individual from a tester line that is used as a parent in a sexual cross. Typically, the tester parent is unrelated to and genetically different from the individual to which it is crossed. A tester is typically used to generate F1 progeny when crossed to individuals or inbred lines for phenotypic evaluation.

The phrase “topcross combination” refers to the process of crossing a single tester line to multiple lines. The purpose of producing such crosses is to determine phenotypic performance of hybrid progeny; that is, to evaluate the ability of each of the multiple lines to produce desirable phenotypes in hybrid progeny derived from the line by the tester cross.

The terms “variety” or “cultivar” mean a group of similar plants that by structural or genetic features and/or performance can be distinguished from other varieties within the same species.

Crop means wheat, maize (corn), rice, sunflower, soybean, tomato, or any plant or plants grown for their food (whether for animal feed or human consumption) or fiber.

Ground seeds, flour, and similar terms refer to whole seeds which have been subject to mechanical disruption and/or pulverization, whether at room temperatures or sub-freezing temperatures. Examples include burr or blade grinding, mill grinding, and mortar and pestle grinding, among others.

High-throughput refers to the processing of multiple samples simultaneously or in rapid succession or both. For example, the instant invention is capable of processing 24 samples simultaneously, which is considered high-throughput. Similarly, processing 48 or 96 samples simultaneously is also considered high-throughput. Additionally, processing one sample individually, or a small number of samples (e.g., eight or less) simultaneously is not considered high-throughput.

Low speed centrifugation means centrifugation at speeds less than 4000×g. The unit “×g” is equivalent to G-forces. In conventional mitochondrial isolation, the prior art teaches the use of high speed centrifugation, e.g., 17,000×g or higher, is necessary to precipitate the mitochondria in order for the mitochondria to be suitable for downstream processes, such as DNA isolation and genotyping.

Seed, kernel, grain, and similar terms, as used herein, refers to a mature plant ovule capable of being sowed and germinated into a plant. For some species, the seed comprises an embryo and endosperm. It may also comprise a seed coat (i.e., a pericarp). Other seeds, e.g., soybean or sunflower, may not comprise an endosperm. Preferably, the seeds used in the instant invention are substantially free of seed chip sampling, endosperm removal, or any other form of individual sampling or modification. The seeds of the instant invention may be from any seed-propagated plant, including but not limited to maize, wheat, and soybean.

Sample plate, sampling plate, sampling block, microwell, microplate, and the like refer to plates comprising at least four wells arrayed in a grid. In one embodiment, the sample plate comprises sample wells arranged in an A×B format, wherein A and B are perpendicular axes, and the number of wells along the A axis can be greater than, less than, or equal to the number of wells along the B axis. In one embodiment, the number of wells along the A axis or B axis is at least 2. In one embodiment, the number of wells along the A axis or B axis is between 2 and 15. In one aspect, the plate comprises 24, 48, or 96 wells in total. In one embodiment, one of the sample wells is connected to another sample well by a frangible region. In one embodiment, the sample plate comprises a base comprising a docking portion for securing the sample plate to a corresponding docking portion of a plate frame holder.

### DETAILED DESCRIPTION

The invention pertains to a method of obtaining plant mitochondria from dry seeds, comprising: (a) obtaining a plurality of dry seeds; (b) grinding the plurality of dry seeds into a powder; (c) sampling from the powder of step (b) a sample and contacting the sample with a homogenization buffer and optionally incubating the contacted sample; (d) centrifuging the contacted sample of step (c) at a speed sufficient to precipitate nuclei and cell debris, thus obtaining a supernatant comprising plant mitochondria; and (e) treating the supernatant of step (d) with a concentration of DNase; wherein the supernatant comprising plant mitochondria is suitable for downstream processes. In one embodiment, the mitochondria are used for mitochondrial DNA (“mtDNA”) extraction. In another, the dry seeds are wheat, barley, corn, rice, sunflower, or other crop plant seed. In yet another, the plant mitochondria are wheat mitochondria.

The invention particularly pertains to a high-throughput method of obtaining plant mitochondria from a plurality of bulked dry seeds, comprising: (a) obtaining a plurality of dry seed bulks; (b) grinding the plurality of dry seed bulks into separate powders; (c) sampling from each of the separate powders of step (b) and placing each sample into an individual well of a sampling plate; (d) adding homogenization buffer to the sample in each well of the sampling plate; (e) centrifuging the sampling plate at a speed sufficient to precipitate nuclei and cell debris, thus obtaining supernatants comprising plant mitochondria; (f) transferring the supernatants to a new sampling plate; and (g) treating the supernatants of step (f) with a concentration of DNase. In one embodiment, the homogenization buffer comprises Tris and sucrose. In one aspect, the the homogenization buffer comprises 50 mM Tris-HCl pH 7.5 and 0.5 M sucrose. In another embodiment, the centrifuging of step (e) is between 2000×g and 4000×g. In yet another embodiment, the sampling plate is a 24-well plate, or a 48-well plate, or a 96-well plate.

The invention also pertains to method of obtaining plant genomic DNA and plant mitochondrial DNA from the same sample of dry seeds, comprising: (a) obtaining a plurality of dry seeds; (b) grinding the plurality of dry seeds into a powder; (c) selecting a sample from the powder of step (b) and contacting the sample with a homogenization buffer; (d) centrifuging the contacted sample of step (c) at a speed sufficient to precipitate nuclei and cell debris, thus obtaining a supernatant comprising subcellular organelles; (e) removing the supernatant of step (d); (f) treating the supernatant of step (d) with a suitable concentration of DNase; (g) extracting organellular DNA from the treated supernatant of step (f); and (f) resuspending the precipitated nuclei and cell debris of step (d) thus obtaining a solution comprising resuspended nuclear DNA; wherein the DNase-treated supernatant of step (f) comprises subcellular plant organelles suitable for organellular genotyping and wherein the solution comprising resuspended nuclear DNA of step (h) is suitable for nuclear genotyping.

### EXAMPLES

The following non-limiting examples show one having ordinary skill in the art how to practice the claimed methods.

### Example 1: Materials

The following materials are used in the claimed method.
1. Homogenization Buffer: 50 mM Tris-HCl, pH 7.5, 0.5 M sucrose. Stored at 4° C.
2. DNase I (100 mg) from Sigma-Aldrich, Inc. (Product Number: 10104159001), stored at 4° C.
3. DNase I dissolving buffer: 50 mM Tris-HCl, pH 7.5, 10 mM CaCl₂, 10 mM MgCl₂, 50% glycerol.
4. DNase I reaction buffer (10×): 500 mM Tris-HCl, pH 7.5, 100 mM MgCl₂, 20 mM CaCl₂.
5. Dellaporta Lysis buffer: 200 mM Tris HCL pH 8.5, EDTA 25 mM, 1% SDS
6. Guanidine Lysis buffer: 4 M Guanidine Thiocyanate, 10 mM Tris.
7. Wash Buffer: 62.5 mM Tris-HCl, pH 7.5, 12.5 mM EDTA, 0.25 M NaCl, 25% ethanol, 25% isopropanol.
8. 7.5M NH4 Acetate.
9. 100% Ethanol (“EtOH”).
10. Isopropanol.
11. 70% EtOH.
12. 1×TE: 10 mM Tris-Cl, pH 8.0, 1 mM EDTA.
13. 24-deep-well sample plate, with four steel beads (at 3/16″ diameter) added to each well, and an appropriate mat, such as a silicon mat cover.
14. 96-well half-height plate.
15. 250-μ1 and 1000-μl and wide orifice tips.

Reasonable substitutions can be made to the above list, and the person having ordinary skill in the art will be aware of such reasonable substitutions. Likewise, slight modifications to the above materials, and the person of ordinary skill in the art will be aware of these modification. For example, guanidine lysis buffer may comprise 4 M Guanidine isothiocyanate (47.2 g/100 ml), 25 mM sodium acetate, pH 6.0, and 1 mM EDTA. See doi:10.1101/pdb.rec431, Cold Spring Harb. Protoc. 2006.

To prepare the DNase for use, start with 100 mg lyophilized DNase and add 40 mL DNase I dissolving buffer, mixing gently. The final concentration of DNase is approximately 5 U/μL. Aliquot 1.0 mL DNase solution into 1.5 mL tubes and store at −20° C.

### Example 2: Seed Flour Sample Preparation

For each bulk of seeds, sample 300 seeds and grind into a fine powder with an appropriate grinder, keeping the flour at 4° C. It is important to ensure that the powder is very fine and to avoid overheating during grinding. The seeds may be any seed, but particularly wheat or barley seed.

Obtain a 24-deepwell sample plate preloaded with steel beads. From each sample of seed flour, subsample into a well approximately 0.3 g of flour using an appropriate sampling tool such as a measuring spoon. Subsamples may be done in singles, duplicates, triplicates, or more. Seal the plate with the mat until ready to add the homogenization buffer.

Once ready, remove the mat and add 3.0 mL homogenization buffer to each well. Resecure the mat and place plate (or plates, if more than one) on an orbital shaker at approximately 300 rpm for approximately 15 minutes. Optionally, one may also use a magnetic plate to dislodge the steel beads and assist mixing the flour and homogenization buffer. Take care not to shake too fast as mitochondria are fragile.

Centrifuge the plate(s) for approximately 20 minutes at low speed (approximately 4000 rpm or 3220×g) at 4° C. in an appropriate device, such as the EPPENDORF® 5810R Refrigerated Centrifuge. Note that if an oil layer and floating particles are observed on the top of or in the supernatant, a second centrifugation is necessary. In this case, carefully transfer 1.4 mL supernatant into a new 24-well plate and perform a second centrifugation at 4000 rpm at 4° C. for approximately 10 minutes.

Remove the mat and carefully transfer 600 μL supernatant (total, using wide orifice pipet tips) to a 96-well half-height plate. Take special care not to touch the pellet in order to avoid nuclear DNA contamination. If subsamples are in duplicate, supernatant from two 24-deepwell plates is combined into one 96-well half-height plate. Store the supernatant, which contains the mitochondria, at 4° C. until ready to progress to the next step.

Prepare the DNase treatment cocktail. The preparation in Table 1 is sufficient for 96 reactions.

TABLE 1
DNase treatment cocktail recipe.

| Components | 1 rxn (ul) | 100 rxn/one 96-well plate (ul) |
|---|---|---|
| 10x DNase buffer | 20 | 2000 |
| DNase I (5 U/μl) | 15 | 1500 |
| Homogenization buffer | 65 | 6500 |
| Total volume | 100 | 10,000 |

Aliquot 100 μL DNase cocktail into each well of a 96-well half-height plate. With wide orifice pipette tips, carefully add 100 μL of the stored supernatant containing the mitochondria into the aliquots of DNase cocktail. Do not touch the well side with the tips; add the supernatant to the middle-bottom of the wells. Slowly pipet up and down three-five times to mix the reaction. Seal the plate with a mat or adhesive plastic film. Centrifuge for approximately 1 minute at approximately 400 rpm at room temperature to ensure collection of well's contents into the bottom of the well. Place the plate on an orbital shaker and shake for approximately 5 minutes at approximately 300 rpm to further mix the reaction. Incubate the plate for approximately 1 hour at 37° C.

At this point, the practitioner has obtained plant mitochondria substantially free of genomic plant DNA from the nucleus.

### Example 3: Guanidine Thiocyanate Lysis of Mitochondria and Isopropanol Precipitation of mtDNA

From the plate containing the plant mitochondria, remove mat or film and add 200 μL lysis buffer to each well to lyse the mitochondria. Reseal the plate and shake on an orbital shaker at approximately 600 rpm for approximately 10 minutes. Centrifuge for 1 minute at 4000 rpm at room temperature.

Add 300 μL Isopropanol into each wells in above plate. Seal the plate with mat and shake on an orbital shaker at approximately 600 rpm for approximately 10 minutes. Centrifuge for approximately 20 minutes at 4000 rpm, either at room temperature or chilled.

Remove mat and discard supernatant by gently inverting the plates or using vacuum aspiration. Add 500 μL per well of 70-80% ethanol to each well. Replace mat and shake for approximately 5 minutes with 600 rpm on an orbital shaker. Centrifuge for approximately 10 minutes at 4000 rpm, either at room temperature or chilled.

Remove mat and discard supernatant. Place the plates upside down on paper towel to absorb liquid as much as possible, allowing the plates to dry for approximately 20 minutes to allow residual ethanol to completely evaporate.

Add 80 μL 1×TE buffer to each well. Place plates on shaker table for a minimum of 1 hour at room temperature, preferably overnight at room temperature.

Centrifuge plates for approximately 5 minutes at 4000 rpm, either at room temperature or chilled. Optionally, one may transfer 70 μl per well to a new, labeled 96-well plate. Store the mitochondrial DNA plates at 4° C. or −20° C. until needed. Prior to use in PCR reactions, centrifuge plates for 5 minutes at 4000 rpm, in order to ensure all liquid is collected at the bottom of the well.

### Example 4: Guanidine Thiocyanate Lysis and mtDNA Isolation with Magnetic Beads

From the plate containing the plant mitochondria, remove mat or film and add 200 μL lysis buffer to each well to lyse the mitochondria. Reseal the plate and shake on an orbital shaker at approximately 600 rpm for approximately 10 minutes. Centrifuge for 1 minute at 4000 rpm at room temperature.

Add 6 μL paramagnetic beads (“PMPs” or “magnetic beads” or “mag beads”) to the side wall of each well, taking care not to touch the solution so as to avoid cross-contamination. If a higher yield of mtDNA is required, increase the PMP volume up to 10 μL per well. Mix the lysis solution and PMPs by pipetting up and down several times. Allow the mtDNA to bind to the PMPs by incubating for at least 5 minutes at room temperature, preferably on an orbital shaker at 400 rpm.

Place the plate on a magnetic plate and allow the PMPs to migrate to the corners of the wells. Aspirate the liquid with a vacuum evacuator, being careful not to aspirate the beads.

Add 400 μL wash buffer or simply 70%-80% EtOH to each well, preferably using multichannel pipette, and mix by pipetting up and down several times or by rotating on an orbital shaker at 400 rpm for 3-5 minutes.

Again place the plate on a magnetic plate and allow the PMPs to migrate to the corners of the wells. Aspirate the liquid with a vacuum evacuator, being careful not to aspirate the beads. Remove the plate from magnet and allow beads to air dry for approximately 15 minutes, or until beads are just dry. The beads are ready once there is no longer a detectable alcohol odor and the beads have turned a lighter shade of brown. Note, take care not to let beads dry for too long.

To elute the mtDNA from the now-dry beads, add 100 μL to each well and mix by pipetting up and down or by rotating on an orbital shaker at 400 rpm for 5-10 minutes.

Place a magnet under the plate and allow the solution to clear. Transfer 90 μL solution containing mtDNA from each well into a new plate. Seal the plate and store at 4° C. for −20° C. until needed. Prior to use, centrifuge the plates for 1-2 minutes at 4000 rpm.

### Example 5: Mitochondria Lysis with Dellaporta Buffer and mtDNA Precipitation with Isopropanol

From the plate containing the plant mitochondria, remove the cover and add 300 μL Dellaporta lysis buffer to each well to lyse the mitochondria. Re-cover and invert the plate a few times to help mix the lysate, and/or optionally shake on an orbital shaker at 600 rpm for approximately 10 minutes. This can help obtain high yield of DNA. Centrifuge for approximately 2 minutes at 4000 rpm at room temperature.

Remove the cover and add 200 μL 7.5M NH4 Acetate to each well. Re-secure the cover and invert the plate a few times to help mix the lysate, and/or optionally shake on an orbital shaker at 600 rpm for approximately 10 minutes. Centrifuge for 15 minutes at 4000 rpm.

During centrifugation, prepare precipitation plates by adding 300 μL isopropanol to a new 96-well plate. Transfer 450 μL per well of supernatant from the spun plate to corresponding wells in the new 96-well plate containing isopropanol. Seal the plate with a lid and gently mix by inverting the plate several times. Centrifuge for 20 minutes at 4000 rpm.

Remove the lid and carefully invert the plates to discard supernatant, or remove the supernatant using vacuum aspiration. Add 500 μL per well of 70% ethanol to each well. Replace the lid and shake for approximately 5 minutes with 600 rpm on an orbital shaker. Centrifuge for 10 minutes at 4000 rpm to pellet the mtDNA. Remove the lid and discard the supernatant. Allow plates to dry for a minimum of 1-2 hours, or as long as overnight, to allow residual ethanol to evaporate.

Add 100 μL 1×TE elution buffer to each well. Seal the plates and place them on an orbital shaker table rotating at approximately 400-600 rpm for a minimum of 1 hour or overnight at room temperature. To assist in breaking up the DNA pellet for a more complete resuspension, the plates be gently inverted or pulsed on a vortex machine after approximately 30 minutes.

Centrifuge plates for approximately 15 minutes at 4000 rpm. Transfer 90 μl per well to a new 96-well plate Cover and store mtDNA plates at 4° C. or −20° C. until needed. Prior to use in PCR reactions, centrifuge plates for 5 minutes at 4000 rpm.

### Example 6: Detecting and Distinguishing mtDNA and gDNA

To detect the presence of wheat mtDNA, real-time PCR (“rtPCR”) reactions were run. rtPCR is well-known in the general state of the art. The primers listed in Table 2 were used to detect wheat mtDNA. The sequence of the amplicon produced is also included.

TABLE 2
Wheat mtDNA primer sequences

| | |
|---|---|
| Forward | CCACCATTTCTCCTGCTTGAA |
| | (SEQ ID NO: 1) |
| Reverse | GTCGAGTGGTCTCAGTTGGAGAT |
| | (SEQ ID NO: 2) |
| Probe | TET-CTCGTTCAATCCATAAACACGT |
| | GCAATCC-BHQ1 |
| | (SEQ ID NO: 3) |
| Amplicon | GTCGAGTGGTCTCAGTTGGAGATGGG |
| | ATTGCACGTGTTTATGGATTGAACGA |
| | GATTCAAGCAGGAGAAATGGTGG |
| | (SEQ ID NO: 4) |

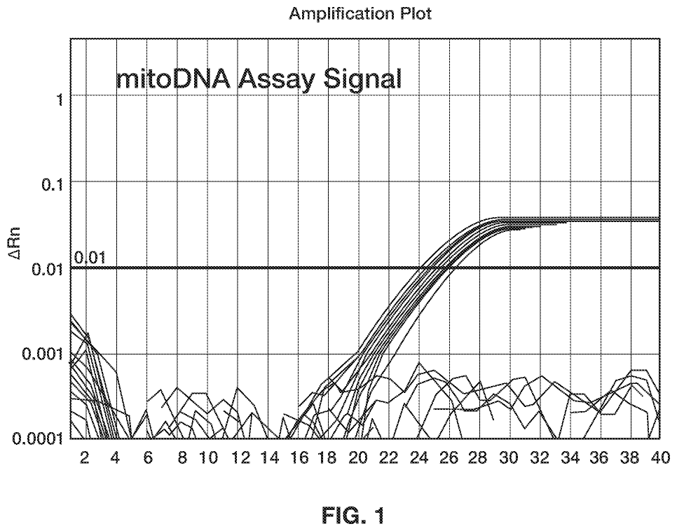

Results of rtPCR reactions showing the presence of wheat mtDNA are shown in FIG. 1.

To detect the presence of contaminant wheat genomic DNA, further rtPCR reactions were run. The primers listed in Table 3 were used to detect contaminant wheat gDNA. The sequence of the amplicon produced is also included.

TABLE 3
Wheat nuclear gDNA primer sequences

| | |
|---|---|
| Forward | CAAGGACGCCGAATTCAAGA |
| | (SEQ ID NO: 5) |
| Reverse | CGAAGAAGGTGCCCTTGAGA |
| | (SEQ ID NO: 6) |
| Probe | TET-CCACCCGATGAACTTC |
| | CTGAACGAGA-BHQ1 |
| | (SEQ ID NO: 7) |
| Amplicon | CAAGGACGCCGAATTCAAGACCCACCC |
| | GATGAACTTCCTGAACGAGAGGACTCT |
| | CAAGGGCACCTTCTTCG |
| | (SEQ ID NO: 8) |

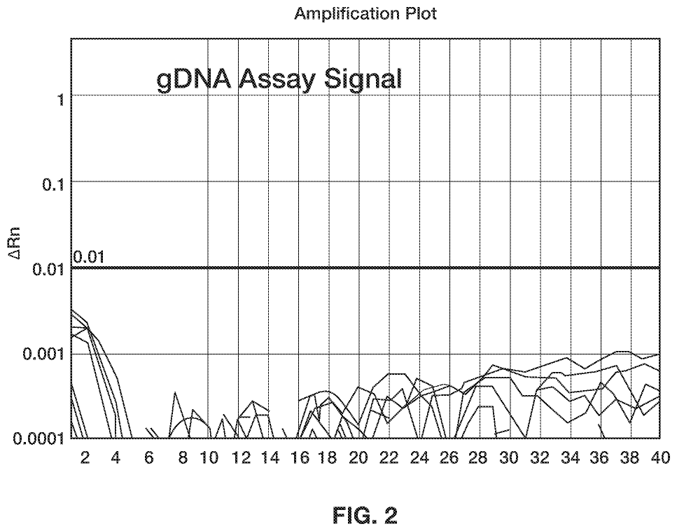

Results of rtPCR reactions showing the absence of wheat nuclear gDNA are shown in FIG. 2.

These results show that high quality mitochondrial DNA was extracted from whole seeds in a high-throughput manner without contamination by the seed's genomic DNA.

What is claimed is:

1. A method of obtaining plant mitochondria from dry seeds, comprising:
a. obtaining a plurality of dry seeds;
b. grinding the plurality of dry seeds into a powder;
c. sampling from the powder of step (b) a sample and contacting the sample with a homogenization buffer and optionally incubating the contacted sample;
d. centrifuging the contacted sample of step (c) at a speed sufficient to precipitate nuclei and cell debris, thus obtaining a supernatant comprising plant mitochondria; and
e. treating the supernatant of step (d) with a concentration of DNase;
wherein the supernatant comprising plant mitochondria is suitable for downstream processes.

2. The method of claim 1, wherein the mitochondria are used for mitochondrial DNA (“mtDNA”) extraction.

3. The method of claim 1, wherein the dry seeds are wheat, barley, corn, rice, sunflower, or other crop plant seed.

4. The method of claim 1, wherein the downstream process is genotyping or genetic purity testing.

5. The method of claim 1, wherein the plant mitochondria are wheat mitochondria.

6. A high-throughput method of obtaining plant mitochondria from a plurality of bulked dry seeds, comprising:
a. obtaining a plurality of dry seed bulks;
b. grinding the plurality of dry seed bulks into separate powders;
c. sampling from each of the separate powders of step (b) and placing each sample into an individual well of a sampling plate;
d. adding homogenization buffer to the sample in each well of the sampling plate;
e. centrifuging the sampling plate at a speed sufficient to precipitate nuclei and cell debris, thus obtaining supernatants comprising plant mitochondria;
f. transferring the supernatants to a new sampling plate; and
g. treating the supernatants of step (f) with a concentration of DNase.

7. The method of claim 6, wherein the homogenization buffer comprises Tris and sucrose.

8. The method of claim 7, wherein the homogenization buffer comprises 50 mM Tris-HCl pH 7.5 and 0.5 M sucrose.

9. The method of claim 6, wherein the centrifuging of step (e) is between 2000×g and 4000×g.

10. The method of claim 6, wherein the sampling plate is a 24-well plate, or a 48-well plate, or a 96-well plate.

11. A method of obtaining plant genomic DNA and plant mitochondrial DNA from the same sample of dry seeds, comprising:
a. obtaining a plurality of dry seeds;
b. grinding the plurality of dry seeds into a powder;
c. selecting a sample from the powder of step (b) and contacting the sample with a homogenization buffer;
d. centrifuging the contacted sample of step (c) at a speed sufficient to precipitate nuclei and cell debris, thus obtaining a supernatant comprising subcellular organelles;
e. removing the supernatant of step (d);
f. treating the supernatant of step (d) with a suitable concentration of DNase;
g. extracting organellular DNA from the treated supernatant of step (f); and
h. resuspending the precipitated nuclei and cell debris of step (d) thus obtaining a solution comprising resuspended nuclear DNA;
wherein the DNase-treated supernatant of step (f) comprises subcellular plant organelles suitable for organellular genotyping and wherein the solution comprising resuspended nuclear DNA of step (h) is suitable for nuclear genotyping.
